(19) United States
(12) Patent Application Publication
(10) Pub. No.: US 2010/0305074 A1
(43) Pub. Date: Dec. 2, 2010

(54) NIACIN-BASED PHARMACEUTICAL COMPOSITIONS

(76) Inventor: H. Thomas Hight, Kennesaw, GA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/594,605

(22) PCT Filed: Apr. 4, 2008
(86) PCT No.: PCT/US08/59425

§ 371 Date: Aug. 17, 2010

Related U.S. Application Data
(60) Provisional application No. 60/921,727, filed on Apr. 4, 2007.
(60) Provisional application No. 61/011,302, filed on Jan. 16, 2008.
(60) Provisional application No. 61/063,484, filed on Feb. 4, 2008.
(60) Provisional application No. 61/072,489, filed on Mar. 31, 2008.

(51) Int. Cl. A61K 31/455; A61K 31/519; A61K 31/616; A61K 31/495; A61P 17/04; A61P 29/00; A61P 3/10

(52) U.S. Cl. 514/161; 514/356; 514/262.1; 514/255.04; 514/342

(57) ABSTRACT

The disclosure relates generally to niacin-based pharmaceutical compositions that include at least one pharmaceutical agent capable of treating a niacin-induced side-effect. Accordingly, one aspect of this disclosure is a pharmaceutical composition for delivering niacin to a patient in need thereof, wherein the composition comprises a therapeutic dose of niacin and a therapeutically effective dose of at least one pharmaceutical agent capable of reducing an adverse side-effect of niacin in the patient, and wherein the pharmaceutical agent is delivered to the patient jointly with the niacin, preferably as a single dosage pill or tablet.

### RELATED APPLICATIONS

This application claims priority to provisional U.S. Provisional Patent Applications Ser. Nos. 60/921,727 filed Apr. 4, 2007; 61/011,302 filed Jan. 16, 2008; 61/063,484 filed Feb. 4, 2008; and ______ filed Mar. 31, 2008, the contents of all of which are hereby expressly incorporated herein by reference.

### FIELD OF THE DISCLOSURE

Embodiments of this disclosure describe a family of niacin containing compositions designed to facilitate broader use of niacin by mitigating some of its side effects, while maximizing the benefits of lipid therapy for patients with lipid disorders.

### BACKGROUND

Cardiovascular disease is the biggest killer in the industrialized world. If the risk of myocardial infarction, stroke and sudden death could be reduced by 50% or more, many lives would be saved, and many more would be changed.

Niacin (nicotinic acid; 3-pyridinecarboxylic acid) is used therapeutically to treat lipid disorders, as it lowers total cholesterol, LDL cholesterol, and LDL particle number, and it raises total HDL and HDL2b sub-particle levels. Niacin, used in combination with other lipid medications, has been shown to lower the incidence of cardiovascular events (heart attacks, strokes, and sudden death) by 60-90% in men with low HDL and coronary artery disease (HATS trial, N Engl J Med 345:1583-1592) and to decrease progression of atherosclerosis. Lipid treatment strategies that raise HDL while also lowering cholesterol have been shown to be more effective at reducing cardiovascular event rates, compared to strategies that only lower cholesterol (Superko and King, Circulation. 2008;117:560-568). By incorporating niacin therapy as part of our cardiovascular prevention strategy, dramatic reductions in heart attacks, strokes and sudden death can be realized.

In spite of these amazing attributes, niacin is not as widely used today as it should be. Niacin therapy is complicated by several noxious side effects, any one of which can prompt patients to quit taking niacin, even after having been on it for years. Two types of side effects limit the use of niacin: acute phase side effects and chronic phase side effects. The compositions of this disclosure address both acute phase and/or chronic phase side effects.

The major acute phase side effect is flushing, or niacin-induced vasodilatation, which has been shown to be due to mast cell prostaglandin release in skin. Flushing has been addressed in for example U.S. Pat. No. 6,469,035. One of the methods to decrease the incidence, severity and duration of prostaglandin-mediated niacin-induced vasodilatation is the concomitant use of anti-inflammatories (see U.S. Pat. No. 6,469,035 and Patent Application 2008/0050429) taken prior to the niacin, or as part of a combination pill with niacin. Aspirin has been used for this purpose.

Although pruritis has been previously described as a component of the prostaglandin-mediated flush response to niacin, there is evidence that at least some aspect of the pruritis experienced as the niacin flush is histamine-mediated.

Anti-inflammatories, namely Non-Steroidal Anti-Inflammatory Drugs (NSAIDs) are useful in preventing or reducing niacin-induced flushing. The problem with using NSAIDs for this purpose is that they are associated with a higher incidence of gastrointestinal problems (McCarty D M. Gastroenterology 1989;96:662, Hawkey C BMJ 1990;300:278), which limit their usefulness in preventing niacin-induced flushing. There is a need, therefore, for an effective niacin single-dose tablet that effectively prevents pruritis and flushing while also protecting the gastrointestinal tract from NSAID-induced complications.

The acute phase flushing is prevented by a combination of niacin with an NSAID and a gastrointestinal protecting agent. Acute phase pruritis is prevented by additionally including a heteroclyclic histamine-1 receptor antagonist (hereafter an H-1 blocking agent, or H-1 blocker).

In contrast to the acute phase prostaglandin-mediated flush reaction, which is usually self-limited and resolved with repetitive niacin exposure, the chronic phase side effects tend to last much longer, and can last for as long as the patient takes niacin. There are six chronic phase side-effects treated by this disclosure: niacin-induced hyperglycemia, hyperuricemia, generalized pruritis, rash, generalized abdominal pain, and epigastric abdominal pain.

It well known to those medical practitioners of the art of niacin therapy: that many patients started on niacin by their physicians sooner or later abort niacin therapy. They stop niacin for two reasons: the noxious side effects and lack of convenience. Although there are some patients with side effects who may stay on the prescribed medications if each and every side effect can be eliminated by taking an additional pill, most patients want a simple easy to take pill. Most will not tolerate taking additional pills to take away the side effects of the niacin. Consequently, they do not persist with their niacin therapy.

### SUMMARY

The disclosure relates generally to niacin-based pharmaceutical compositions that include at least one pharmaceutical agent capable of treating a niacin-induced side-effect. Accordingly, one aspect of this disclosure is a pharmaceutical composition for delivering niacin to a patient in need thereof, wherein the composition comprises a therapeutic dose of niacin and a therapeutically effective dose of at least one pharmaceutical agent capable of reducing an adverse side-effect of niacin in the patient, and wherein the pharmaceutical agent is delivered to the patient jointly with the niacin.

In embodiments of this aspect of the disclosure, the pharmaceutical composition may further comprise a pharmaceutically acceptable carrier or excipient.

In the embodiments of the compositions of this aspect of the disclosure, the niacin may be configured for immediate release, intermediate release or sustained release.

In the embodiments, the niacin-induced side-effect treated by the pharmaceutical agent is at least one of the group consisting of flushing, hyperglycemia, pruritis (itching), a gastrointestinal side effect and hyperuricemia.

In the embodiments, the at least one pharmaceutical agent may be a non-steroidal anti-inflammatory, a blood glucose lowering agent; an antihistamine, allopurinol, or a combination thereof.

In one embodiment of the disclosure, the niacin and the at least one pharmaceutical agent are configured for time-release to the patient.

In the embodiments of the disclosure, the composition is in the form of a pill or tablet, or a liquid.

In one embodiment of the disclosure, the at least one pharmaceutical agent is a non-steroidal anti-inflammatory drug.

In one embodiment of the disclosure, the pharmaceutical composition may further comprise a therapeutic amount of a pharmaceutical agent capable of reducing an adverse side-effect on the patient from the non-steroidal anti-inflammatory drug.

In one embodiment of the disclosure, the pharmaceutical agent capable of reducing an adverse side-effect on the patient from the non-steroidal anti-inflammatory drug is a proton pump inhibitor or an H2 receptor blocker, and wherein the pharmaceutical agent capable of reducing an adverse side-effect on the patient from the non-steroidal anti-inflammatory drug may be pantoprazole or omeprazole.

In embodiments of the disclosure, the non-steroidal anti-inflammatory drug may be selected from the group consisting of aspirin, meloxicam, indomethacin, naproxen, naproxen sodium, flurbiprofen, oxaprozin, sulindac, diflunisal, ibuprofen, piroxicam, nabumetone, salsalate, choline magnesium trisalycilate, etalodac, ketoprofen, ketorolac tromethamine, doclofenac potassium, diclofenac sodium, tolmetin sodium, tramadol and celecoxib.

In other embodiments of the disclosure the pharmaceutical composition may further comprise an HMG CoA reductase inhibitor. In various embodiments the HMG CoA reductase inhibitor may be selected from lovastatin, fluvastatin, atorvastatin, simvastatin, rosuvastatin, velostatin, fluindostatin and pravastatin sodium or a mixture thereof.

In one embodiment of the disclosure, the at least one pharmaceutical agent is an anti-histamine.

In this embodiment of the disclosure, the anti-histamine may be a non-sedating anti-histamine selected from the group consisting of fexofenadine, loratidine, desloratidine, terfenadine, astemazole, and cetrizine, or a sedating anti-histamine selected from the group consisting of promethazine, diphenhydramine, hydroxyzene, chlorpheniramine maleate, chlortripalon, brompheniramine, dexchlorpheniramine, cyproheptadine, azatadine, meclozine, dimenhydranate, and alimemazine.

In other embodiments of the disclosure, the at least one pharmaceutical agent is effective in treating a hyperglycemic side-effect of niacin on the patient, wherein the at least one pharmaceutical agent may be a biguanide or a TZD. In these embodiments the TZD may be pioglitazone or rosiglitazone.

Another aspect of the disclosure is a method of reducing niacin-induced flushing while protecting the gastrointestinal tract in a patient receiving niacin for a lipid disorders, comprising administering to the recipient patient a therapeutic composition comprising niacin, a non-steroidal anti-inflammatory drug, and a gastrointestinal protecting agent.

In embodiments of this aspect of the disclosure, the therapeutic composition may further comprise a cholesterol-lowering agent, wherein the agent is a statin.

Another aspect of the disclosure is a method of treating niacin-induced hyperglycemia in patients who are not diabetic comprising delivering to the patient a single therapeutic dose comprising a therapeutically effective dose of niacin and a therapeutically effective dose of a pharmaceutical agent capable of lowering blood sugar levels with a blood sugar-lowering agent in one pill or the like.

Yet another aspect of the disclosure is a method of treating niacin-induced chronic phase histamine-mediated niacin side effects by combining niacin with a histamine blocking agent in one pill or the like.

Yet another aspect of the disclosure is a method of preventing niacin-induced acute pruritis and flushing by combining niacin with an H-1 blocking agent, an NSAID, and a GI protecting agent in one pill or the like.

### DETAILED DESCRIPTION OF THE DISCLOSURE

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein cited references”), as well as each document or reference cited in each of the herein-cited references (including any manufacturer's specifications, instructions, etc.), is hereby expressly incorporated herein by reference.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

Embodiments of the present disclosure will employ, unless otherwise indicated, techniques of synthetic organic chemistry, biochemistry, biology, molecular biology, and the like, which are within the skill of the art. Such techniques are explained fully in the literature.

The methods of this disclosure are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for.

It must be noted that, as used in the specification and the appended claims, the singular forms “a,” “an,” and “the” include plural referents unless the context clearly dictates otherwise. Thus, for example, reference to “a support” includes a plurality of supports.

In this specification and in the claims that follow, reference will be made to a number of terms that shall be defined to have the following meanings unless a contrary intention is apparent. As used herein, the following terms have the meanings ascribed to them unless specified otherwise. In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### DEFINITIONS

In describing and claiming the disclosed subject matter, the following terminology will be used in accordance with the definitions set forth below.

The term “composition” as used herein encompasses a product comprising the specified ingredients in the specified amounts, as well as any product which results, directly or indirectly, from combinations of the specified ingredients in the specified amounts.

The term “treat”, “treating”, and “treatment” are an approach for obtaining beneficial or desired clinical results. Specifically, beneficial or desired clinical results include, but are not limited to, alleviation of symptoms, diminishment of extent of disease, stabilization (e.g., not worsening) of disease, preventing spread of disease, preventing the disease from occurring in a patient that may be predisposed to the disease but does not yet experience or exhibit symptoms of the disease (prophylactic treatment), delaying or slowing of disease progression, amelioration or palliation of the disease state, and remission (partial or total) whether detectable or undetectable. In addition, “treat”, “treating”, and “treatment” can also mean prolonging survival as compared to expected survival if not receiving treatment.

“Pharmaceutically acceptable salts” include, but are not limited to, the acid addition salts of compounds of the present disclosure (formed with free amino groups of the peptide) which are formed with inorganic acids (e.g., hydrochloric acid or phosphoric acids) and organic acids (e.g., acetic, oxalic, tartaric, or maleic acid). Salts formed with the free carboxyl groups may also be derived from inorganic bases (e.g., sodium, potassium, ammonium, calcium, or ferric hydroxides), and organic bases (e.g., isopropylamine, trimethylamine, 2-ethylamino-ethanol, histidine, and procaine).

An “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

The terms “effective amount” and therapeutically-effective amount” as used herein means that amount of a compound, material, or composition comprising a compound or composition of the present disclosure, and which is effective for producing a desired therapeutic effect, biological or medicinal response in a tissue system, animal or human that is being sought by a researcher, veterinarian, medical doctor or other clinician, which includes alleviation of the symptoms of the disease or disorder being treated including, but not limited to, a reduction in dyslipemia or a reduction in a side-effect due to an administered pharmaceutical agent.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or an encapsulating material such as liposomes, polyethylene glycol (PEG), PEGylated liposomes, nonoparticles and the like, involved in carrying or transporting the subject compositions or thereapeutic agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term ‘patient’ as used herein refers to any mammal that may take a lipid altering or anti-inflammatory agent for any of the cardiovascular uses described herein. Administration of lipid-lowering compositions of the present disclosure includes both self-administration and administration to the patient by another person. Humans are the preferred ‘patients’ for the compositions and methods described herein.

The compositions according to the present disclosure are preferably formulated in a unit dosage form. The term ‘unit dosage form’ as used herein refers to physically discrete units suitable as unitary dosages for human patients and other mammals with each unit containing a predetermined quantity of active material calculated to produce the desired therapeutic effect in association with suitable pharmaceutical carriers or excipients. In the case of the HMG-CoA reductase inhibitors, for example, a single daily unit dose may be preferable for most of the statins. In the case of high-dose niacin compositions, the daily dose may be divided into 2 or 3 unit doses that are taken at different times throughout the day, or as in a once a day form that is a controlled release form or even controlled release in multiple unit does, so as to reduce adverse side-effects as much as possible. In a significant percentage of patients, however, the side-effects are great enough even with controlled niacin release, that additional pharmaceutical intervention is desirable. Since the adverse side-effects in question can even result in the patient needing treatment if the effects are severe, intervention is preferable at the time the niacin is delivered to the patient.

The compositions of the present disclosure may be administered in admixture with suitable pharmaceutical diluents, excipients, or carriers that vary depending on the format of the unit dosage form and consistent with prior art and conventional pharmaceutical practices. Since α-lipoic acid and corosolic acid both have low solubility in water, 50 to 200 mg of a carrier such as lecithin and/or oil such as rice bran or olive oil may be added to the composition to increase bioavailability of lipid-soluble agents.

To prepare the pharmaceutical compositions of this invention, one or more compounds of the present disclosure as the active ingredient is intimately admixed with a pharmaceutical carrier according to conventional pharmaceutical compounding techniques, which carrier may take a wide variety of forms depending of the form of preparation desired for administration, e.g., oral. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included.

The pharmaceutical compositions herein will contain, per unit dosage unit, e.g., tablet, capsule, powder, injection, suppository, teaspoonful and the like, of from about 50-100 mg and may be given at a dosage of from about 0.5-5.0 mg/kg/day, preferably from about 1.0-3.0 mg/kg/day. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules for oral administration. Alternatively, the composition may be presented in a form suitable for sustained or daily administration. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.1 to about 5000 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating dyslipidemia and the side effects induced by niacin described in the present disclosure may also be carried out using a pharmaceutical composition comprising any of the compounds as defined herein and a pharmaceutically acceptable carrier. The pharmaceutical composition may contain between about 0.01 mg and 5000 mg, preferably about 50 to 1000 mg, of the compound, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixers, emulsions, and suspensions.

Advantageously, compounds of the present invention may be administered in a single daily dose, or the total daily dosage may be administered in divided doses of two, three or four times daily. Furthermore, compounds for the present invention can be administered orally in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

In the present application, the term “pharmaceutically acceptable salts” shall mean non-toxic salts of the compounds employed in this disclosure which are generally prepared by reacting the free acid with a suitable organic or inorganic base. Examples of such salts include, but are not limited to benzoate, bicarbonate, sodium, calcium, acetate, laurate, malate, maleate, succinate, tannate, tartrate, benzenesulfonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrobromide, hydrochloride, hydroxynapthoate, iodide, isothionate, lactate, lactobionate, mandelate, mesylate, methylbromide, methylnitrate, methylsulfate, mucate, napsylate, nitrate, oleate, oxalate, pamoate, palmitate, panthothenate, phosphate/diphosphate, polygalacturonate, potassium, salicylate, stearate, subacetate, teoclate, tosylate, and valerate.

Effective doses of the compounds of the present disclosure may be ascertained by conventional methods. The specific dosage level required for any particular patient will depend on a number of factors, including severity of the condition being treated, the route of administration and the weight of the patient. In general, however, it is anticipated that the daily dose (whether administered as a single dose or as divided doses) will be in the range 0.01 to 6000 mg per day, more usually from 1 to 500 mg per day, and most usually from 10 to 200 mg per day. Expressed as dosage per unit body weight, a typical dose will be expected to be between 0.0001 mg/kg and 60 mg/kg, especially between 0.01 mg/kg and 7 mg/kg, and most especially between 0.15 mg/kg and 2.5 mg/kg. Although the dosage used will vary depending on the clinical goals to be achieved, a suitable dosage range is one which provides up to about 10 mg to about 1,000 mg or about 10,000 mg of an agent that reduces a level of dyslipidemia and/or effectively treats dyslipidemia, in the case of niacin or a side-effect in a patient induced by niacin.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

For niacin, the dose is about 100 to 6000 mg per does taken 1, 2, 3 or 4 times per day (e.g., immediate-release, intermediate-release, or sustained-release) for a total of about 100 to 6,000 mg of niacin per day.

It is of course understood that the compounds of the present disclosure relate to all optical isomers and stereo-isomers at the various possible atoms of the molecule.

### DISCUSSION

The present disclosure relates to pharmaceutical compositions or methods of delivery thereof intended for the joint delivery of niacin with at least one pharmaceutical agent that prevents or reduces adverse side-effects induced by niacin in a patient. Niacin (nicotinic acid, Vitamin B3) has been used for many years to treat lipid disorders. Unfortunately, while very effective in treating dyslipidemia, many patients experience unpleasant and occasionally unpleasant side effects like flushing, increased blood sugars and gastrointestinal side effects. The extreme nature of these side-effects often leads such patients, who would otherwise benefit from receiving niacin, to forego or discontinue such medication. The present disclosure, therefore, encompasses a group of novel pharmaceutical compositions that usefully combine a number of other medications with the niacin, the particular pharmaceutical agents of the compositions being selected for their abilities to reduce the level of the side effects induced by niacin. This makes it easier for the patient to tolerate the medication and increase the acceptance of niacin as a treatment for dyslipidemia. In particular, the present disclosure encompasses pharmaceutical compositions comprising niacin, a non-steroidal anti-inflammatory drug (NSAID) and/or another pharmaceutical agent intended to reduce adverse gastrointestinal side-effects due to the NSAIDs. By combining the niacin with the other pharmaceutical agents, the latter can begin to act in the patient to prevent the onset of adverse niacin effects, or at the least to significantly reduce the effects to a level where they become tolerable to the patient.

As mentioned above, although pruritis has been previously described as a component of the prostaglandin-mediated flush response to niacin, there is evidence that at least some aspect of the pruritis experienced as the niacin flush is histamine-mediated. Therefore there is a need for an agent that blocks not only the prostaglandin-mediated flush response, but that also blocks the concomitant histamine-mediated pruritis.

By formulating the disclosed compositions to be in single pill or tablet forms, the patient is relieved of the burden of taking multiple medications as a number of pills, and the insurance co-pay costs are reduced. All of these benefits encourage the patient to remain with the prescribed course of treatment and not discontinue, with possibly long-term or fatal consequences to their health.

### Acute Phase Niacin-Induced Side Effects

The major acute phase niacin-induced side effect is flushing, or vasodilatation, which is primarily prostaglandin mediated. Compositions of the present disclosure, typically in a pill form, may comprise, but are not limited to, niacin, an NSAID and a gastrointestinal protecting agent for prevention of gastrointestinal problems caused by the NSAID. In light of the clinical observation that niacin-induced generalized pruritis is histamine-mediated; an embodiment of the disclosure may also include an H1 blocking agent.

A triple combination of a gastrointestinal protecting agent with the NSAID and niacin can prevent the niacin flush reaction while also avoiding NSAID-induced gastrointestinal complications. (See Example 1) Without the gastrointestinal protective effect of this disclosure, administering an NSAID to prevent flushing is not a practical option for a patient.

A quadruple combination of niacin with an H-1 blocker, an NSAID, and a GI protecting agent can prevent the pruritis and flushing with safety to the GI tract. (See Example 2).

Niacin (nicotinic acid, or 3-pyridinecarboxylic acid) used in the compositions herein disclosed can be in the form of an immediate release, an intermediate release (e.g., SLO-NIACIN™) or a sustained release (e.g., NIASPAN™) form. Suitable NSAIDs for use in the compositions of the disclosure include, but are not limited to, indomethacin, celicoxib, meloxicam, piroxicam, nabumetone, salsalate, choline magnesium trisalicylate, diflunisal, aspirin, naproxen, naproxen sodium, flurbiprofen, oxaprozin, sulindac, ibuprofen, ketoprofen, ketorolac tomethamine, etolodac, diclofenac sodium, dicolfenac potassium, tolmetin sodium, and any salts thereof.

Niacin-induced flushing can occur several hours after taking a sustained release form of niacin such as NIASPAN™. Therefore it is useful for the compositions of the present disclosure to include both an immediate release form of an NSAID such as indomethacin (INDOCIN™) and/or a sustained release form of the NSAID such as INDOCIN.SR™. (See Example 3)

Gastrointestinal agents are included for the purpose of protecting the gastrointestinal tract from the side effects triggered by the NSAID. One drug from either of two different gastrointestinal drug subclasses may be used, namely a Proton Pump Inhibitor (PPIs) such as, but not limited to, omeprazole, omeprazole magnesium, pantoprazole sodium, lansoprazole, esomeprazole magnesium, rabeprazole sodium, leminoprazole, timoprazole, tenatoprazole, disulprazole, RO-18-5362 and IY 81149. The second subclass consists of H-2 blockers, such as cimetidine, famotidine, nizatidine and ranitidine.

### Chronic Phase Niacin-Induced Side Effects

One chronic phase niacin-induced side effect frequently observed is hyperglycemia. Although hyperglycemia can occur in any patient treated with niacin, those who seem to be at particular risk are pre-diabetic patients with insulin resistance or glucose intolerance, and obese patients who gain weight while taking niacin.

Diabetes and pre-diabetes involve unique cardiovascular risk factors, separately distinguishable from lipid-related risk factors like cholesterol and HDL. If carbohydrate-metabolism related risk is worsened (i.e., hypoglycemia occurs) as a result of treating the lipids with niacin, then patients may not be receiving optimum care.

Diabetic patients are usually already on diabetic medications, the doses of which can be adjusted for niacin-induced hyperglycemia accordingly. However, non-diabetic patients on niacin therapy who develop fasting hyperglycemia may also need to be treated with a blood sugar lowering agent, in order to safely continue niacin therapy. (See Example 4).

Insulin resistance has been associated with an increased risk of premature closure of coronary stents. Patients who have high cardiovascular risk and are also at high risk for developing niacin-induced hyperglycemia may also benefit from the combination of niacin with an insulin sensitizing agent. (See Example 5).

The compositions of the present disclosure may incorporate any of two classes of diabetic medications: the biguanide metformin in combination with niacin, or a peroxisome proliferator-activated receptor-γ agonist (PPAR-γ agonist, also called thioglitazone, or TZD) to achieve those ends. Pioglitazone hydrochloride and rosiglitazone maleate are of particular usefulness in the compositions of the present disclosure. These blood sugar lowering drugs may be used in combination with niacin to prevent or treat hyperglycemia. Any or all of these three drugs may be in the form of immediate release or sustained release formulations. In addition, other compounds such as an NSAID and a gastrointestinal drug may be included with the niacin and diabetic medication to ameliorate the niacin-induced flushing. All of the components of the compositions herein disclosed are preferably, but not only, delivered to the patient as a single pill or tablet.

Generalized pruritis, or itching of the skin, is one of the most common chronic phase side effects of niacin therapy. Chronic phase pruritis may be distinguished from the flushing caused by niacin in the acute phase. Chronic pruritis is experienced by patients who take immediate-release niacin, intermediate-release niacin, and sustained-release niacin. Pruritis has been described as occurring acutely, as part of the initial niacin-induced skin vasodilatation called flushing. Chronic phase pruritis, however, occurs in the absence of skin vasodilatation. There is no visible rash or hot sensation or flushing of the skin. Patients may present with excoriations and scarring of the skin due to scratching.

Whereas the acute phase skin vasodilatation tends to resolve after repetitive niacin therapy, chronic phase pruritis may last for years, or for as long as the patient takes niacin. The acute phase skin vasodilatation has been found to be primarily prostaglandin-mediated. While not wishing to be bound by any one theory, however, chronic phase pruritis is most likely histamine-mediated. Histamine-mediated niacin induced pruritis may be effectively treated by the heterocyclic histamine-1 receptor antagonists, namely H1 blocking agents, combined with niacin and other agents in the compositions according to the present disclosure. (See Example 6).

The H1 blocking agent suitable for use in the compositions of the present disclosure may be selected from the following two subgroups: (a) the non-sedating anti-histamines: fexofenadine, loratidine, desloratidine, terfenadine, astemazole, or cetrizine and (b) the older group of antihistamines considered to be sedating, such as promethazine, diphenhydramine, hydroxyzene, chlorpheniramine maleate, chlortripalon, brompheniramine, dexchlorpheniramine, cyproheptadine, azatadine, meclozine, dimenhydranate, and alimemazine. Either of these subgroups may be paired with immediate release or intermediate release or sustained release niacin in a combination pill that may also optionally includes a non-steroidal anti-inflammatory and/or a gastrointestinal drug.

It is anticipated that when niacin is taken at bedtime (as is recommended by the manufacturers of NIASPAN™ for example), the H1 blocking agent may be from the sedating subclass. When the niacin is being taken by the patient during daytime hours, in a bid or tid dose, the non-bedtime doses preferably include the non-sedating type of H1 blocker. Since the niacin-induced histamine-mediated pruritis may occur several hours after taking the sustained release form of niacin, a preferred embodiment would contain both an immediate release and a sustained or intermediate release form of the H1 blocker.

The third chronic phase side effect treated by the compositions of the present disclosure is hyperuricemia, a known complication of niacin therapy. For patients who develop hyperuricemia while on niacin, the combination of niacin with the uricosuric agent allopurinol can prevent gouty attacks, while allowing the niacin patient to continue therapy without interruption. The effects of combining niacin and allopurinol in a combination of the present disclosure are described in Example 7 below.

Another recognized chronic phase niacin-induced side effect is generalized abdominal discomfort of a cramping or a dull aching quality, sometimes described as waxing and waning, and sometimes associated with vague diffuse abdominal tenderness on examination (but not epigastric tenderness). The compositions of the present disclosure incorporating H-1 blockers (non-sedating for daytime administration, sedating for bedtime dosing) are effective in reducing or eliminating niacin induced histamine-mediated generalized abdominal discomfort. In another embodiment, an H-2 blocking agent for treatment of niacin induced histamine-mediated epigastric discomfort may be combined with niacin in the compositions of the present disclosure. Suitable H2 receptor blockers include, but are not limited to, cimetidine, famotidine, or ranitidine.

Sustained-release niacin is currently being used in combination with statin drugs, such as lovastatin (ADVICOR™, Abbott Pharmaceuticals) or simvastatin (SIMCOR™, Abbott Pharmaceuticals), or ezetimibe (ZETIA™Schering/Plough). It is also contemplated that the compositions of the present disclosure may combine sustained-release niacin with both an NSAID, and a gastrointestinal protectant drug intended to counteract the adverse side effects due to the NSAID, as well as with a statin such as lovastatin, pravastatin, simvastatin, atorvastatin, or rosuvastatin calcium. It is further contemplated that the compositions herein disclosed are formulated as a single dosage pill or tablet, with the doses of the individual compounds being tailored to the needs of a patient.

All formulations of the disclosed compositions may utilize an immediate release form of niacin, an intermediate release form (such as that used in SLO-NIACIN™) or a sustained release delivery system for the nicotinic acid, or niacin (such as that used in NIASPAN™). There are 6 groups of agents that may be used in combination with niacin: Group 1 (NSAIDs), Group 2 (gastrointestinal protecting agents), Group 3 (blood sugar-lowering agents), Group 4 (antihistamines), Group 5 (uricosurics), and Group 6 drugs (cholesterol-lowering agents).

Group 1 agents: for prevention of flushing: The non-steroidal anti-inflammatory agents that may be combined with either immediate release niacin or sustained release niacin include (but are not limited to): aspirin, meloxicam, indomethacin, naproxen, naproxen sodium, flurbiprofen, oxaprozin, sulindac, diflunisal, ibuprofen, piroxicam, nabumetone, salsalate, choline magnesium trisalycilate, etalodac, ketoprofen, ketorolac tromethamine, doclofenac potassium, diclofenac sodium, tolmetin sodium, tramadol and celecoxib.
Group 2 agents: for prevention of non-steroidal induced gastrointestinal side effects: The Group 2 gastrointestinal agents in combination with niacin and the nonsteroidal include (but are not limited to): a proton pump inhibitor (such as, omeprazole), or sucralfate, or misoprostol, or an H2 receptor blocker such as cimetidine, famotidine, or ranitidine. Either one of these gastrointestinal drugs may be used as the third drug in the combination.
Group 3 agents for prevention of hyperglycemia: Blood glucose lowering drugs include (but are not limited to) two groups, the biguanides and the TZDs such as rosiglitazone and pioglitazone. This drug may be combined with niacin, either in immediate release or intermediate or sustained release form.
Group 4 agents, for prevention of the histamine-mediated side effects of niacin: Group 4 agents, designed to prevent the histamine-mediated side effects of niacin, consists of two subgroups, the first being the non-sedating anti-histamines: fexofenadine, loratidine, desloratidine, terfenadine, astemazole, or cetrizine. Also included is the second subgroup consisting of the older group of antihistamines considered to be sedating, such as promethazine, diphenhydramine, hydroxyzene, chlorpheniramine maleate, chlortripalon, brompheniramine, dexchlorpheniramine, cyproheptadine, azatadine, meclozine, dimenhydranate, and alimemazine. Either one of these agents may be paired with immediate release or intermediate release or sustained release niacin, and may also be included in a combination pill with niacin, and/or a non-steroidal, and/or a gastrointestinal drug.
Group 5 agents: for the prevention and treatment of hyperuricemia:_Allopurinol is the only agent in this category. Niacin therapy is sometimes complicated by hyperuricemia. In that setting, the combination of niacin with allopurinol can prevent gouty attacks, while allowing the niacin patient to continue therapy without interruption. There are some patients who need niacin therapy, but who have a past history of gout or hyperuriemia. This embodiment is also effective in those patients, right from the start.
Group 6 agents: for the enhancement of the lipid-lowering effects of niacin: Group 6 agents consist of two classes: HMG-CoA Reductase Inhibitors (statins) and ezetimbe (ZETIA™). Statins include (but are not limited to): lovastatin, simvastatin, rosuvastatin calcium, atorvastatin calcium, and pravastatin sodium, including salts thereof.
When niacin therapy is complicated by hyperglycemia, a Group 2 agent is used. When niacin therapy is complicated by histamine-mediated pruritis or abdominal pain, then a Group 3 agent is used.
If a patient has prostaglandin-mediated flushing and histamine-mediated gastrointestinal side effects, a combination pill with niacin, a Group 1 drug, a Group 2 drug and a Group 4 drug may be used.

When an enhanced lipid effect is desired, a Group 6 agent may be added to the niacin. EZETIMIBE™ may be part of a combination pill with any one of the statins. This family of niacin-containing pharmaceuticals might contain multiple agents, from any of the Groups, including two from Group 5. Some non-limiting examples are: a pill containing each of these ingredients: niacin, a NSAID, a Group 2 agent, an agent from Group 3 and two agents from Group 6 (for the patient who has glucose intolerance and a serious hyperlipidemia like familial Fredrickson Type IIA, who is starting niacin therapy). Another embodiment may be a combination pill containing each of these ingredients: niacin, a Group 2 drug, a Group 5 drug, and a Group 6 drug (for the seriously hypercholesterolemic patient who has been taking niacin for two months, having had complete resolution of the flushing, but then develops hyperuricemia and histamine-mediated epigastric discomfort). Another embodiment may be a combination pill containing niacin, two Group 3 drugs (a sedating H1 blocker for use at bedtime and a non-sedating H1 blocker for use in the morning), and a Group 2 drug (for the patient who needs 2000 mg, but who has the flush reaction at a niacin dose of 1500 mg and so is unable to tolerate more than 1000 mg of niacin at one time, therefore needing to take a bid dose of niacin, morning and evening, but is suffering from niacin-induced histamine-mediated pruritis, and so uses a non-sedating H1 blocker combination tablet in the morning, and a sedating H1 blocker combination tablet in the evening, and has developed fasting hyperglycemia).

Combinations of these different agents is possible. Drug delivery system can be used to deliver embodiments of the present disclosure. Immediate release niacin may be combined with a non-sedating and/or a sedating antihistamine from Group 4, and with a sustained release drug from Group 3. A sustained release niacin formulation may be combined with both an immediate release NSAID and a sustained release NSAID, and with a PPI (a gastrointestinal Group drug).

Accordingly, one aspect of this disclosure is a pharmaceutical composition for delivering niacin to a patient in need thereof, wherein the composition comprises a therapeutic dose of niacin and a therapeutically effective dose of at least one pharmaceutical agent capable of reducing an adverse side-effect of niacin in the patient, and wherein the pharmaceutical agent is delivered to the patient jointly with the niacin.

In embodiments of this aspect of the disclosure, the pharmaceutical composition may further comprise a pharmaceutically acceptable carrier or excipient.

In the embodiments of the compositions of this aspect of the disclosure, the niacin may be configured for immediate release, intermediate release or sustained release.

In the embodiments, the niacin-induced side-effect treated by the pharmaceutical agent is at least one of the group consisting of flushing, hyperglyceremia, pruritis (itching), a gastrointestinal side effect and hyperuricemia.

In the embodiments, the at least one pharmaceutical agent may be a non-steroidal anti-inflammatory, a blood glucose lowering agent; an antihistamine, allopurinol, or a combination thereof.

In one embodiment of the disclosure, the niacin and the at least one pharmaceutical agent are configured for time-release to the patient.

In the embodiments of the disclosure, the composition is in the form of a pill or tablet, or a liquid.

In one embodiment of the disclosure, the at least one pharmaceutical agent is a non-steroidal anti-inflammatory drug.

In one embodiment of the disclosure, the pharmaceutical composition may further comprise a therapeutic amount of a pharmaceutical agent capable of reducing an adverse side-effect on the patient from the non-steroidal anti-inflammatory drug.

In one embodiment of the disclosure, the pharmaceutical agent capable of reducing an adverse side-effect on the patient from the non-steroidal anti-inflammatory drug is a proton pump inhibitor or an H2 receptor blocker, and wherein the pharmaceutical agent capable of reducing an adverse side-effect on the patient from the non-steroidal anti-inflammatory drug may be pantoprazole, omeprazole or a prostaglandin E1 analog.

In embodiments of the disclosure, the non-steroidal anti-inflammatory drug may be selected from the group consisting of aspirin, meloxicam, indomethacin, naproxen, naproxen sodium, flurbiprofen, oxaprozin, sulindac, diflunisal, ibuprofen, piroxicam, nabumetone, salsalate, choline magnesium trisalycilate, etalodac, ketoprofen, ketorolac tromethamine, doclofenac potassium, diclofenac sodium, tolmetin sodium, tramadol and celecoxib.

In other embodiments of the disclosure the pharmaceutical composition may further comprise an HMG CoA reductase inhibitor. In various embodiments the HMG CoA reductase inhibitor may be selected from lovastatin, fluvastatin, atorvastatin, simvastatin, rosuvastatin, velostatin, fluindostatin and pravastatin sodium or a mixture thereof.

In one embodiment of the disclosure, the at least one pharmaceutical agent is an anti-histamine.

In this embodiment of the disclosure, the anti-histamine may be a non-sedating anti-histamine selected from the group consisting of fexofenadine, loratidine, desloratidine, terfenadine, astemazole, and cetrizine, or a sedating anti-histamine selected from the group consisting of promethazine, diphenhydramine, hydroxyzene, chlorpheniramine maleate, chlortripalon, brompheniramine, dexchlorpheniramine, cyproheptadine, azatadine, meclozine, dimenhydranate, and alimemazine.

In other embodiments of the disclosure, the at least one pharmaceutical agent is effective in treating a hyperglycemic side-effect of niacin on the patient, wherein the at least one pharmaceutical agent may be a biguanide or a TZD. In these embodiments the TZD may be pioglitazone or rosiglitazone.

Another aspect of the disclosure is a method of reducing niacin-induced flushing while protecting the gastrointestinal tract in a patient receiving niacin for a lipid disorders, comprising administering to the recipient patient a therapeutic composition comprising niacin, a non-steroidal anti-inflammatory drug, and a gastrointestinal protecting agent.

In embodiments of this aspect of the disclosure, the therapeutic composition may further comprise a cholesterol-lowering agent, wherein the agent is a statin.

Another aspect of the disclosure is a method of treating niacin-induced hyperglycemia in patients who are not diabetic comprising delivering to the patient a single therapeutic dose comprising a therapeutically effective dose of niacin and a therapeutically effective dose of a pharmaceutical agent capable of lowering blood sugar levels with a blood sugar-lowering agent in one pill or the like.

Yet another aspect of the disclosure is a method of treating niacin-induced chronic phase histamine-mediated niacin side effects by combining niacin with an histamine blocking agent in one pill or the like.

Unless otherwise defined, all technical and scientific terms used herein have the same meaning as commonly understood by one of ordinary skill in the art of molecular biology. Although methods and materials similar or equivalent to those described herein can be used in the practice or testing of the present disclosure, suitable methods and materials are described herein. All publications, patent applications, patents, and other references mentioned herein are incorporated by reference in their entirety. In addition, the materials, methods, and examples are illustrative only and are not intended to be limiting.

The following examples are provided to describe and illustrate, but not limit, the claimed disclosure. Those of skill in the art will readily recognize a variety of non-critical parameters that could be changed or modified to yield essentially similar results.

### EXAMPLES

### Example 1

Thirteen patients, who initiated sustained-release niacin therapy using Aspirin having 81 mg for prevention of flushing, continued to have debilitating flushing. They were then treated with a more potent NSAID, together with a PPI to prevent GI complications. Instead of aborting their niacin therapy, twelve were able to continue. The flushing was abolished or was made tolerable, with no NSAID-related GI complications.

### Example 2

Three patients were initiated on niacin therapy using a combination of an NSAID with an H-1 blocking agent. All three had no symptoms at all.

### Example 3

Seven of the thirteen patients listed above in Example 1 who had debilitating flushing were treated with the sustained release NSAID Indocin SR™ 75 mg. Six patients were able to continue taking niacin. One stopped the Indocin SR™ due to GI complaints. Two eventually stopped niacin due to other chronic phase side effects.

### Example 4

Eight male patients with niacin-induced hyperglycemia, but without diabetes, were followed, ranging in ages from 38 to 80. All were told to lose weight. Two did. In one patient who lost weight, the hyperglycemia resolved and the niacin dose was subsequently increased. The other 7 patients suffered from worsening hyperglycemia on niacin therapy. All 7 were offered diabetic medications as an off-label way to neutralize the cardiovascular risk associated with hyperglycemia. Five of them agreed to treatment (three took metformin, two a TZD). Two did not consent to therapy, choosing instead to keep trying to control the hyperglycemia with diet and exercise.

The two who weren't treated with blood sugar lowering agents eventually developed frank Type II Diabetes, and both discontinued niacin when blood sugars were high. Neither has restarted niacin.

The five who agreed to use blood sugar lowering medications have all continued to take niacin. None has developed Diabetes after a minimum of 7 months (average 12, range 7-22 mo) of follow-up. One on a TZD was able to successfully lose weight and then discontinue pioglitazone (15 mg).

### Example 5

A 47yo male with chest pain and two-vessel coronary artery disease was treated with angioplasty and stent placement. Risk factor evaluation revealed stimulated hyperinsulinemia (fasting C-peptide 2.52, 2 hour post 75 gm stimulated C-peptide 12.54) and low HDL-2 level (10, by VAP lab). Niaspan™ was started at 500 mg and increased to 3,000 mg daily. At the same time, he was treated with a glucose-lowering agent. Although he stopped smoking, he gained 46 pounds over 4 years (BMI: 30.5, 4 yr BMI: 37). In spite of his weight gain, he has not developed Type II Diabetes, and has been able to continue niacin therapy, achieving an HDL2b level of 21 (Berkeley HeartLabs) an ApoB level of 53, with HemA1c levels below 6.0. He is currently on pioglitazone (Actos™) 15 mg.

### Example 6

Nine patients with niacin-induced pruritis were treated by recommending an H-1 blocking agent. Of two who decided to stop niacin therapy, one quit due to frustration over “taking too many pills.” The other quit taking niacin after being on 2,000 mg of Niaspan™ daily for 20 months. He has not been willing to try again to take niacin with the H-1 blocker. Seven patients were successful, with resolution of pruritis. For morning dosing, four took cetrizine (Zyrtec™), and one took fexofenadine (Allegra™). For evening dosing, six took diphenhydramine (Benadryl™). All seven were able to continue niacin therapy.

### Example 7

Sixteen patients developed hyperuricemia while on niacin therapy. Two females and 15 males were given allopurinol to control the hyperuricemia (daily dosing ranged from 25 to 200 mg). All were able to continue niacin therapy. The average time between niacin initiation and the onset of hyperuricemia was six months, ranging from 1 to 15 months.

We claim:

1. A pharmaceutical composition for delivering niacin to a patient in need thereof, wherein the composition comprises a therapeutic dose of niacin and a therapeutically effective dose of at least one pharmaceutical agent capable of reducing an adverse side-effect of niacin in the patient, and wherein the pharmaceutical agent is delivered to the patient jointly with the niacin.

2. The pharmaceutical composition of claim 1, further comprising a pharmaceutically acceptable carrier or excipient.

3. The pharmaceutical composition of claim 1, wherein the niacin is configured for immediate-release, intermediate-release, or sustained-release.

4. The pharmaceutical composition of claim 1, wherein the niacin-induced side-effect treated by the pharmaceutical agent is at least one of the group consisting of flushing, hyperglyceremia, pruritis (itching), a gastrointestinal side effect and hyperuricemia.

5. The pharmaceutical composition of claim 1, wherein the at least one pharmaceutical agent is a non-steroidal anti-inflammatory; a blood glucose lowering agent; an antihistamine; allopurinol; or a combination thereof.

6. The pharmaceutical composition of claim 1, wherein the niacin and the at least one pharmaceutical agent are configured for time-release to the patient.

7. The pharmaceutical composition of claim 1, wherein the composition is in the form of a pill or tablet, or a liquid.

8. The pharmaceutical composition of claim 1, wherein the at least one pharmaceutical agent is a non-steroidal anti-inflammatory drug.

9. The pharmaceutical composition of claim 8, further comprising a therapeutic amount of a pharmaceutical agent capable of reducing an adverse side-effect on the patient from the non-steroidal anti-inflammatory drug.

10. The pharmaceutical composition of claim 9, wherein the pharmaceutical agent capable of reducing an adverse side-effect on the patient from the non-steroidal anti-inflammatory drug is a proton pump inhibitor or an H2 receptor blocker.

11. The pharmaceutical composition of claim 10, wherein the pharmaceutical agent capable of reducing an adverse GI side-effect on the patient from the non-steroidal anti-inflammatory drug is selected from: omeprazole, omeprazole magnesium, pantoprazole sodium, lansoprazole, esomeprazole magnesium, rabeprazole sodium, leminoprazole, timoprazole, tenatoprazole, disulprazole, and combinations thereof.

12. The pharmaceutical composition of claim 8, wherein the non-steroidal anti-inflammatory drug is selected from the group consisting of aspirin, meloxicam, indomethacin, naproxen, naproxen sodium, flurbiprofen, oxaprozin, sulindac, diflunisal, ibuprofen, piroxicam, nabumetone, salsalate, choline magnesium trisalycilate, etalodac, ketoprofen, ketorolac tromethamine, doclofenac potassium, diclofenac sodium, tolmetin sodium, tramadol and celecoxib.

13. The pharmaceutical composition of claim 1, further comprising an HMG CoA reductase inhibitor.

14. The pharmaceutical composition of claim 13, wherein the HMG CoA reductase inhibitor is selected from lovastatin, fluvastatin, atorvastatin, simvastatin, rosuvastatin, velostatin, fluindostatin, ezetimibe, and pravastatin sodium, or a mixture thereof.

15. The pharmaceutical composition of claim 1, wherein the at least one pharmaceutical agent is an anti-histamine.

16. The pharmaceutical composition of claim 15, wherein the anti-histamine is a non-sedating anti-histamine selected from the group consisting of fexofenadine, loratidine, desloratidine, terfenadine, astemazole, and cetrizine, or a sedating anti-histamine selected from the group consisting of promethazine, diphenhydramine, hydroxyzene, chlorpheniramine maleate, chlortripalon, brompheniramine, dexchlorpheniramine, cyproheptadine, azatadine, meclozine, dimenhydranate, and alimemazine.

17. The system of claim 1, wherein the at least one pharmaceutical agent is effective in treating a hyperglycemic side-effect of niacin on the patient.

18. The system of claim 17, wherein the at least one pharmaceutical agent effective in treating a hyperglycemic side-effect of niacin on the patient is a biguanide or a TZD.

19. The system of claim 18, wherein the TZD is pioglitazone or rosiglitazone.

20. A method of reducing niacin-induced flushing while protecting the gastrointestinal tract in a patient receiving niacin for a lipid disorders, comprising administering to the recipient patient a therapeutic composition comprising niacin, a non-steroidal anti-inflammatory drug, and a gastrointestinal protecting agent.

21. The method of claim 20, wherein the therapeutic composition further comprises a cholesterol-lowering agent, wherein the agent is a statin.

22. A method of treating niacin-induced hyperglycemia in patients who are not diabetic comprising delivering to the patient a single therapeutic dose comprising a therapeutically effective dose of niacin and a therapeutically effective dose of a pharmaceutical agent capable of lowering blood sugar levels with a blood sugar-lowering agent in one pill.

23. A method of treating niacin-induced chronic phase histamine-mediated niacin side effects by combining niacin with an histamine blocking agent in one pill.
